(19) United States
(12) Patent Application Publication
(10) Pub. No.: US 2004/0126369 A1
(43) Pub. Date: Jul. 1, 2004

(54) Delivery of peroxide-generating enzymes to the vaginal tract

(76) Inventors: Richard Payne, Roswell, GA (US); Curtis Sayre, Atlanta, GA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/331,955

(22) Filed: Dec. 30, 2002

(51) Int. Cl. A61K 38/44

(52) U.S. Cl. 424/94.4

(57) ABSTRACT

The present invention relates to methods and compositions for intravaginal administration of peroxide-generating enzymes and substrates for promoting the growth of Gram-positive bacilli, inhibiting Gram-negative bacilli, promoting an oxidizing environment, and/or treating or preventing disturbances of the vaginal bacterial flora accompanying the reduction of Gram-positive bacilli or the increase in pathogenic microbes, such as Gram-negative bacilli, resulting in vaginal infections.

### BACKGROUND OF THE INVENTION

Healthy vaginal microenvironments contain hydrogen peroxide and have a pH from about 4 to 5. Predominate organisms within the healthy vagina include lactobacilli and other Gram-positive bacilli; however, pathogenic organisms, such as Candida, also exist at low levels in a normal vaginal environment. The normal environment can be disrupted in many ways, including systemic antibiotics, menopause, or other factors. These disruptions often result in infections, or an increase in the number of pathogenic microbes and a decrease in lactobacilli or other Gram-positive beneficial bacilli. When Gram-positive bacilli are reduced or disappear in the vagina, vaginal pH rises, oxidation decreases, and a disturbance of normal vaginal bacterial flora results from an abnormal increase in Gram-negative bacilli or other pathogenic microbes, including Gram-positive cocci and Gram-negative cocci, which can cause harm to the human body and lead to a range of diseases and symptoms, including bacterial vaginosis.

Options currently available for treatment of reduced Gram-positive bacilli colonization and increased numbers of pathogenic microbes are not particularly effective. For example, antibacterial drugs are used to suppress the growth of Gram-negative bacilli and other abnormal bacteria. However, these drugs may result in significant side effects to the patient. Furthermore, antibacterial drugs may also inhibit the growth of Gram-positive bacilli, resulting in difficulty in reestablishing a healthy vaginal microenvironment. Direct administration of lactobacilli in the vagina is also not very effective, in that it usually does not result in stable colonization. An additional limitation of these two treatments, and many others, is that they simply do not attempt to normalize the vaginal flora growth habitat by maintaining, enhancing or creating acceptable vaginal environmental conditions.

Thus, there is a need in the art for compositions and methods for treating disturbances of the normal vaginal bacterial flora and promoting growth/repopulation of the vaginal tract by Gram-positive bacilli, including lactobacilli.

### SUMMARY OF THE INVENTION

The present invention concerns the discovery that imbalances in the vaginal flora may be effectively treated through the administration of an effective amount of at least one peroxide-generating enzyme, such as a hydrogen peroxide-generating enzyme, and an effective amount of at least one suitable substrate to the vaginal tract (intravaginally) of a mammal, for example, a human. For example, the present invention is directed to the use of peroxide-generating enzymes to produce low doses of peroxide for an extended period of time to help restore and/or maintain a healthy vaginal microenvironment. Accordingly, the present invention provides therapeutic and/or prophylactic compositions and methods for inducing and maintaining an oxidizing environment in the vagina in order to control the growth of microorganisms that cause disturbances of the vaginal flora.

One embodiment of the present invention is a composition for use in the therapeutic or prophylactic treatment of a disturbance of vaginal bacterial flora in mammals including vaginal administration of an effective amount of a composition comprising an enzyme capable of producing peroxide upon administration of said composition to the vagina of said mammal. Additionally, the composition may include a substrate that said enzyme is capable of acting on to produce peroxide and/or a carrier for delivery to the vagina. For example, the substrate includes an oxidizable substrate, while the enzyme is an oxidoreductase enzyme, the action of which yields peroxide, such as hydrogen peroxide. The carrier may be any number of materials, including a tampon, a syringe-like applicator, or a liquid.

Another embodiment of the present invention is a method for treating or preventing a disturbance of vaginal bacterial flora in mammals comprising vaginal administration of an effective amount of a composition comprising an enzyme capable of producing peroxide upon administration of said composition to the vagina of said mammal. Additionally, the composition may include a substrate that said enzyme is capable of acting on to produce peroxide and/or a carrier for delivery to the vagina.

One embodiment of the present is an article for use in the therapeutic or prophylactic treatment of a disturbance of vaginal bacterial flora in mammals including an enzyme capable of producing peroxide, the article being adapted for use in the vagina.

### BRIEF DESCRIPTION OF THE DRAWINGS

FIG. 1 is a graphical representation of the hydrogen peroxide production resulting from alcohol oxidase (1.0 Unit in 0.5 ml) activity with carboxymethylcellulose (0.25% w/v) over time (at pH 6.3, no initial peroxide added, agitated at 37° C. in normal atmosphere).

FIG. 2 is a graphical representation of the inhibition of E. coli growth with varying concentrations of hydrogen peroxide over time (LBG media, pH 6.9 and agitated at 37° C. in normal atmosphere).

FIG. 3 is a graphical representation of the inhibition of C. albicans growth with varying concentrations of hydrogen peroxide over time (Peptone media, pH 6.5 and agitated at 37° C. in normal atmosphere).

FIG. 4 is a graphical representation of the effect of pH on hydrogen peroxide production. Depicted is hydrogen peroxide production resulting from alcohol oxidase activity with carboxymethylcellulose as a function of pH (with no initial peroxide added, after agitation for 8 hours at 37° C. in normal atmosphere).

FIG. 5 is a graphical representation of the effect of initial hydrogen peroxide (H₂O₂) on H₂O₂ production. Depicted is hydrogen peroxide production resulting from alcohol oxidase activity with carboxymethylcellulose as a function of initial hydrogen peroxide added (at pH 6.3, after agitation for 8 hours at 37° C. in normal atmosphere).

FIG. 6 depicts a tampon.

FIG. 7 depicts a syringe-like applicator.

### DETAILED DESCRIPTION OF THE INVENTION

Vaginal infections or disturbances of vaginal bacterial flora are often the result of an imbalance of vaginal microflora. Typically, the pathogenic agent is one that normally exists in low levels in healthy vaginal systems. When an event occurs (e.g., systemic antibiotics or menopause) that reduces the number of lactobacilli and other beneficial microorganisms that normally predominate in healthy systems, harmful microorganisms multiply and induce a pathogenic state in and/or surrounding the vagina. One method by which beneficial bacteria (e.g., lactobacilli) in the vaginal tract suppress the growth of pathogenic organisms is through the production of hydrogen peroxide. Hydrogen peroxide, while not harmful to lactobacilli or vaginal tissue, is harmful to pathogenic organisms and works to keep their numbers low. Thus, administration of one or more peroxide-generating enzymes, for example hydrogen peroxide-generating enzymes, to the vaginal tract is useful to inhibit growth of pathogenic microbes, allowing the lactobacilli to proliferate and dominate the vaginal microenvironment, and thus, establish a healthy vaginal environment.

The methods and compositions of the present invention may be useful for prophylactic and/or therapeutic treatment of disturbances of vaginal microflora. As used herein, the phrase “disturbances of vaginal microflora” means an imbalance in the normal homeostasis of vaginal flora, such as a decrease in Gram-positive bacilli and/or an increase in Gram-negative bacilli as compared to a healthy vaginal environment. Specific disturbances or imbalances in the vaginal microflora for which the methods or compositions of the invention may be useful include, but are not limited to, fungal (e.g., yeast infection), bacterial (e.g., bacterial vaginosis), viral or parasitic (e.g., trichomonas) infections. The infections include those characterized/diagnosed as vaginitis, vaginal candidiasis, and vaginosis. Examples of some of the microorganisms that cause such infections include microorganisms of the genus Candida, particularly C. albicans and C. tropicalis, and T. glabrata, Gardneralla (vaginalis), various mixed anaerobic bacteria and Peptostreptococcus bacteria. These vaginal infections may result in a pathological discharge from the vagina, great discomfort to a female patient, and/or serious medical consequences if left untreated.

As used herein, the term “enzyme” means a molecule that acts as a catalyst in chemical reactions. The term “substrate” means a material or substance on which an enzyme acts to produce a desired product.

As used herein, the phrase “effective amount” means an amount that is effective in treating a particular infection (e.g., a disturbance of the vaginal microflora) and is determinable by a person of skill in the art. For example, an effective amount of enzyme and/or substrate is that amount which is sufficient to inhibit the growth of pathogenic microbes (e.g., Gram-negative bacilli).

The phrase “pharmaceutically acceptable” is used herein to mean that the material so described can be used for treatments in or on humans or other mammals without causing ill effects, such as toxicity or blistering.

As used herein, the term “peroxide” means any compound containing the divalent ion —O—O—, including, but not limited to, a compound containing an oxygen-oxygen single bond of the type: R₁—O—O—R₂ For example, a compound of the type R₁—O—O—R₂, wherein R₁ and R₂ may be hydrogen, alkyl groups, ketones, R-carbonyls, aromatic groups, or any combination thereof.

As used herein, a “bioadhesive” is a material that adheres to a live or freshly killed biological surface, such as a mucous membrane (e.g., a mucoadhesive) or skin tissue. Bioadhesives useful in the present invention include, but are not limited to, the commercially available materials sold under the designation polycarbophil by A. H. Robins Co. of Richmond, Va., and CARBOPOL® “Ex55” also known as CARBOPOL® 976 and NOVEON All® by B.F. Goodrich Chemical Co. of Cleveland, Ohio. Suggested mucoadhesives include, but are not limited to, poly(acrylic acid) (PAA), poly(methacrylic acid), poly(vinyl pyrrolidone), cellulose derivatives, chitosan, alginate, pectin, and gelatin.

As used herein, the term “therapeutic” means to cure or restore to health, while the term “prophylactic” means to preserve health or to prevent or contribute to the prevention of disease.

I. Enzymes and Substrates

In the methods and compositions of the invention, a variety of enzyme-substrate combinations can be used. The enzymatic component of the therapeutic and/or prophylactic compositions of the present invention comprises one or more peroxide-generating enzymes including, but not limited to, lipoxygenase, prostaglandin synthase, or oxidoreductase.

Lipoxygenase and prostaglandin synthase act on arachidonate with oxygen to produce non-hydrogen peroxide structures. For example, a reaction catalyzed by lipoxygenase yields 5-HPETE, while a reaction catalyzed by prostaglandin synthase yields prostaglandin G2, both products have peroxide functionality. (Biochemistry, Voet and Voet, Second Edition, John Wiley & Sons, New York, pp. 707-708 (1995).)

In one desirable embodiment, the enzyme is an oxidoreductase enzyme. Oxidoreductase enzymes are capable of oxidizing one or more substrates to produce hydrogen peroxide (e.g., glucose oxidase, which converts glucose and oxygen into gluconic acid and hydrogen peroxide; or alcohol oxidase which catalyzes the interaction of a primary alcohol and oxygen to produce hydrogen peroxide). Such enzymes include those enzymes classified according to the Nomenclature Committee of the International Union of Biochemistry and Molecular Biology (NC-IUBMB) as E.C.1.-.-.-enzymes (oxidoreductases), for example, those classified as E.C.1.1.-.-enzymes (acting on the CH—OH group of donors), or those classified as E.C. 1.1.3.-enzymes (with oxygen as acceptor). Illustrative examples of oxidoreductase enzymes and substrates useful in the present invention are set forth below in Table I.

TABLE 1
OXIDOREDUCTASE ENZYMES

| E.C. CLASSI- | | |
|---|---|---|
| FICATION | NAME | SUBSTRATE |
| E.C. 1.1.3.3 | Malate | (S)-malate |
| | Oxidase | |
| E.C. 1.1.3.4 | Glucose | β-D-glucose |
| | Oxidase | |
| E.C. 1.1.3.5 | Hexose | β-D-glucose; also oxidizes D- |
| | Oxidase | galactose, D-mannose, maltose, |
| | | lactose and cellobiose |
| E.C. 1.1.3.6 | Cholesterol | Cholesterol |
| | Oxidase | |
| E.C. 1.1.3.7 | Aryl-Alcohol | An aromatic primary alcohol; |
| | Oxidase | Oxidizes many primary alcohols |
| | | containing an aromatic ring, |
| | | including (2-naphthyl)methanol |
| | | and 3-methoxybenzyl alcohol |
| E.C. 1.1.3.8 | L-Gulonolactone | L-gulono-1,4-lactone |
| | Oxidase | |
| E.C. 1.1.3.9 | Galactose | D-galactose |
| | Oxidase | |
| E.C. 1.1.3.10 | Pyranose | D-glucose; also oxidizes D-xylose, |
| | Oxidase | L-sorbose and D-glucono-1,5- |
| | | lactone, which have the same ring |
| | | conformation and configuration at |
| | | C-2, C-3 and C-4 |
| E.C. 1.1.3.11 | L-Sorbose | L-sorbose; also acts on D-glucose, |
| | Oxidase | D-galactose and D-xylose |
| E.C. 1.1.3.12 | Pyridoxine 4- | Pyridoxine |
| | Oxidase | |
| E.C. 1.1.3.13 | Alcohol | A primary alcohol; acts on lower |
| | Oxidase | primary alcohols and unsaturated |
| | | alcohols |
| E.C. 1.1.3.14 | Catechol | Catechol |
| | Oxidase | |
| E.C. 1.1.3.15 | (S)-2-Hydroxy-Acid | (S)-2-hydroxy acid |
| | Oxidase | |
| E.C. 1.1.3.16 | Ecdysone | Ecdysone |
| | Oxidase | |
| E.C. 1.1.3.17 | Choline | Choline |
| | Oxidase | |

| E.C. | | |
|---|---|---|
| CLASSI- | | |
| FICATION | NAME | SUBSTRATE |
| E.C. 1.1.3.18 | Secondary-Alcohol | A secondary alcohol; acts on |
| | Oxidase | secondary alcohols with five or |
| | | more carbons, and polyvinyl |
| | | alcohols with molecular mass over |
| | | 300 Da |
| E.C. 1.1.3.19 | 4- | (S)-2-hydroxy-2-(4- |
| | Hydroxymandelate | hydroxyphenyl)acetate |
| | Oxidase | |
| E.C. 1.1.3.20 | Long-Chain-Alcohol | Long-chain alcohol; oxidizes long- |
| | Oxidase | chain fatty alcohols, including |
| | | dodecyl alcohol |
| E.C. 1.1.3.21 | Glycerol-3- | sn-glycerol 3-phosphate |
| | Phosphate | |
| | Oxidase | |
| E.C. 1.1.3.22 | Xanthine | Xanthine; also oxidizes |
| | Oxidase | hypoxanthine, some other purines |
| | | and pterins, and aldehydes; |
| | | probably acts on the hydrated |
| | | derivatives of these substrates |
| E.C. 1.1.3.23 | Thiamine | Thiamine |
| | Oxidase | |
| E.C. 1.1.3.24 | L-Galactonolactone | L-galactono-1,4-lactone; acts on |
| | Oxidase | the 1,4-lactones of L-galactonic, |
| | | D-altronic, L-fuconic, D-arabinic |
| | | and D-threonic acids |
| E.C. 1.1.3.25 | Cellobiose | Cellobiose; also oxidizes |
| | Oxidase | cellodextrins, lactose, and, more |
| | | slowly, 4-β-D-glucosyl-D- |
| | | mannose |
| E.C. 1.1.3.26 | Columbamine | Columbamine |
| | Oxidase | |
| E.C. 1.1.3.27 | Hydroxyphytanate | L-2-hydroxyphytanate |
| | Oxidase | |
| E.C. 1.1.3.28 | Nucleoside Oxidase | Inosine; other purine and |
| | | pyrimidine nucleosides (as well as |
| | | 2′-deoxynucleosides) are |
| | | substrates |
| E.C. 1.1.3.29 | N-Acylhexosamine | N-acetyl-D-glucosamine; also acts |
| | Oxidase | on N-glycolylglucosamine, N- |
| | | acetylgalactosamine and, more |
| | | slowly, on N-acetylmannosamine |
| E.C. 1.1.3.30 | Polyvinyl-Alcohol | Polyvinyl alcohol |
| | Oxidase | |
| E.C. 1.1.3.31 | Methanol Oxidase | Methanol; acts on some aliphatic |
| | | alcohols |
| E.C. 1.1.3.37 | D-Arabinono-1, | D-arabinono-1,4-lactone |
| | 4-Lactone Oxidase | |
| E.C. 1.1.3.38 | Vanillyl-Alcohol | Vanillyl alcohol |
| | Oxidase | |
| E.C. 1.1.3.39 | Nucleoside | Adenosine; other purine and |
| | Oxidase | pyrimidine nucleosides, as well as |
| | | 2′-deoxynucleosides and |
| | | arabinosides, are substrates |
| E.C. 1.1.3.40 | D-Mannitol | Mannitol; also catalyses the |
| | Oxidase | oxidation of D-arabinitol and D- |
| | | glucitol (sorbitol) |

In the pathogenic state, the vaginal pH is typically a pH of about 6 to about 7, which is much higher than that of the healthy state, a pH of about 4 to about 4.5. A number of peroxide producing enzymes will produce high levels of peroxide at a pH of around 6 to 7. The activity of these enzymes decreases with decreasing pH. This phenomenon can be used to self-limit the enzymes of the compositions and methods of the present invention. That is, if an enzyme and a substrate are placed into a vaginal system with elevated pH, the enzyme will produce peroxide. This peroxide will inhibit pathogenic microorganisms and allow the beneficial microorganisms to proliferate. As a healthy vaginal homeostasis is reestablished, the pH will drop and the activity of the enzyme will fall. This corresponds with a reduction in the need for peroxide generation, and provides that peroxide generation will decrease at normal, healthy pHs.

In addition, several peroxide-generating enzymes deactivate in the presence of high concentrations of peroxide, e.g., greater than or equal to 0.1% peroxide. This prevents the overproduction of peroxide, and keeps peroxide levels below concentrations harmful to beneficial vaginal bacteria and/or healthy vaginal tissue. Thus, one embodiment of the present invention provides for enzymes that are self-limiting, including enzymes that are self-limiting with respect to pH and the concentration of peroxide in the environment.

Additionally, enzymes useful in the compositions and methods of the present invention may be administered without the administration of a suitable substrate. Substances present in the vagina due to secreted products from the mammal's vaginal cells or products produced from microorganisms present in the vagina may be useful as substrates for the production of peroxide by the enzymes of the compositions and methods of the present invention.

II. Additional Components of the Peroxide-Generating Enzyme System

In addition to an enzyme that catalyzes the production of peroxide and a suitable substrate, the compositions of the instant invention may further comprise at least one other agent effective for use in therapeutic and/or prophylactic treatment of vaginal conditions, such as disturbances of vaginal microflora, or in the alleviation of symptoms thereof. For example, the compositions of the invention may also comprise one or more acids to aid in lowering the pH of the vagina. Suggested acids include, but are not limited to, acetic acid, caproic acid, boric acid, and lactic acid.

Additionally, the compositions of the present invention may also include a topical anesthetic, such as lidocaine hydrochloride or topical steroids, such as corticosteroid, to provide relief from pain or itching.

In another desirable embodiment, compositions of the invention may further include one or more bioadhesive or mucoadhesive agents. One advantage of a composition that includes a bioadhesive agent is that after vaginal administration, the composition will remain in place for several days, providing for long-lasting treatment. Additionally, fewer applications of a composition comprising a bioadhesive agent would be required for full-efficacy. Further, bioadhesives, including thermogelling mucoadhesives, act as a moisturizer while promoting the growth of beneficial bacteria.

The compositions of the invention may also include additional antimicrobial agents, such as antibacterials, including, but not limited to, metronidazole, polymyxin, or aztreonam. The compositions of the invention may also include defensins (antimicrobial peptides) and/or protamine. Further, the compositions of the invention may include antifungals, including but not limited to, ketoconazole, terconazole, itraconazole, or fluconazole.

Compositions useful in the present invention may also contain one or more pharmaceutically acceptable additives that are referred to herein as adjuvants that typically assist in providing extended shelf life and customer acceptance of the methods and compositions of the instant invention. Exemplary adjuvants include, but are not limited to, preservatives, emollients, lubricating oils, emulsifying agents, humectants, coloring agents, fragrance and/or odor providing agents (odorants). The enzyme and/or substrate may be included, for example, in a micelle or liposome, or some other encapsulated form, or administered as a prodrug or in an extended release form to provide a prolonged storage and/or delivery effect (e.g., sustained release).

Suggested preservatives that may be included in compositions of the present invention include, but are not limited to, alcohol, ascorbyl palmitate, benzoic acid, butylated hydroxyanisole, butylated hydroxytoluene, chlorobutanol, ethylenediamine, ethylparaben, ethyl vanillin, glycerin, methylparaben, monothioglycerol, phenol, phenylethyl alcohol, phenylmercuric nitrate, propylparaben, sassafras oil, sodium benzoate, sodium formaldehyde sulfoxylate, sodium metabisulfite, sorbic acid, sulfur dioxide, maleic acid, or propyl gallate.

Suggested emollients that may be included in compositions of the present invention include, but are not limited to, fatty or oleaginous substances including castor oil, sulfated castor oil, cocoa butter, coconut oil, cold cream, corn oil, cottonseed oil, rosewater ointment, combinations of white wax and white petrolatum, combinations of sodium lauryl sulfate, propylene glycol and stearyl alcohol, sesame oil, theobroma oil, myristyl alcohol or shark liver oil.

Typical lubricating agents or oils that may be included in compositions of the present invention include, but are not limited to, petrolatum, white or yellow wax, coca butter, oleic acid, olive oil, jojoba oil, paraffin, starch glycerite, lanolin, hydrophilic petrolatum, mineral oil, cetyl alcohol, glyceryl monostearate, stearic acid, polyethylene glycols, polyoxyl 40 stearate, polysorbate, cholesterol or higher molecular weight lipids. Emollients and lubricants provide products with the appropriate slip, tactile feel and rub-in properties to enhance the ease of usage and to encourage the consumer to use the product as often as needed.

Emulsifying agents are used to produce oil-in-water emulsions. Typical emulsifying agents useful in the compositions of the present invention include, but are not limited to, sodium alginate, acacia, carbomer, sodium carboxymethylcellulose, carrageenan, gelatin, hydroxyethylcellulose, hydroxypropylcellulose, hydroxypropylmethylcellulose, octoxynol-9, oleyl alcohol, polyvinyl alcohol, povidone, bentonite, graphite, magnesium hydroxide sodium lauryl sulfate, sorbitan esters, stearyl alcohol, tragacanth, potassium laurate, polyoxyethylene sorbitan monooleate or xanthan gum.

Humectants that may be included in compositions of the present invention include, but are not limited to, glycerin, propylene glycol, pyrrolidone carboxylic acid, sodium lactate, urea, or certain natural lipid mixtures. Other suggested humectants include, but are not limited to, certain proteins, gelatin, hyaluronic acid, vitamins or some natural ingredients.

III. Dose

A. Dosage Forms

For therapeutic, prophylactic and/or hygienic use, the compositions of the present invention can be administered to the vagina as an article or carrier in the form of a vaginal insert, syringe-like applicator, tablet, suppository, pessary, powder/talc or other solid, solution, liquid, spray, aerosol, pharmaceutically acceptable carrier, douche, ointment, tampon, foam, cream, gel, including a bioadhesive gel (e.g., a mucoadhesive thermogelling composition (see, for example, U.S. application Ser. No. 10/135,805, filed on Apr. 30, 2002, which is incorporated herein by reference)), paste, microcapsules, vaginal sponge, vaginal ring, or controlled, sustained release compositions. Controlled sustained release can be achieved by the addition of time-release additives, such as polymeric structures, microcapsules, matrices, etc., known in the art. The compositions of the invention may also be administered through the use of hot-melt extrusion articles, such as bioadhesive hot-melt extruded film (see, for example, U.S. Pat. No. 6,375,963, which is incorporated herein by reference).

Liquid compositions of the invention can be administered from absorbent materials, such as a tampon or sponge, or as a spray/aerosol (applied to the affected area using a pump-type or aerosol sprayer). The use of a tampon, in which the intravaginal composition of the invention has been incorporated, is advantageous in that it cannot be diluted or carried away by menstrual blood or other vaginal discharge. Providing the composition in the form of a solution, which may initially be provided in a concentrated liquid form, or as a dissolvable powder, tablet or the like requiring the addition of water, saline or other suitable diluents prior to use, enables the composition to be administered as a vaginal douche.

Solid compositions of the invention can be applied by any number of means, including the use of applicators or patient self-insertion. For example, creams, lotions, suppositories, foams, pastes, ointments, gels, tablets, or tampons may be applied to the vagina using an applicator, such as a squeeze-type or plunger-type applicator well known for use in applying vaginal products. Administering the composition as a suppository is advantageous as it provides convenience, ease of application, increased safety and/or neatness. Administering the composition as a cream having low surface tension is advantageous as it provides a uniform wetting action that assists in composition penetration into vaginal crypts and crevices and acts as a moisturizer.

One desirable embodiment provides for compositions of the invention in a syringe-like applicator (20; also known as a plunger-type applicator (see FIG. 7)). For example, a gel, including but not limited to, a bioadhesive gel, containing a substrate, including but not limited to, carboxymethyl cellulose, may be placed into a first chamber (60) of a syringe like applicator (20) and sealed with a barrier (30; the presence of the barrier allows for the enzyme and substrate to be separate, so as not to react, while remaining in the same applicator during assembly, shipping and handling, but will rupture when the user depresses the plunger). An enzyme, including, but not limited to, alcohol oxidase, is then placed in a second chamber (50) of the syringe-like applicator and stabilized for storage, such as by freeze-drying, to aid in shelf-stability. The enzyme chamber is placed in series at the outlet of the gel chamber. At the time of use, the applicator is inserted into the vagina and the plunger (40) is depressed. This force will push the gel through the barrier and through the enzyme chamber, where it rehydrates the enzyme and carries it out of the applicator and into the vagina. Thus, the combination of the enzyme and substrate at the time of use activates the enzyme.

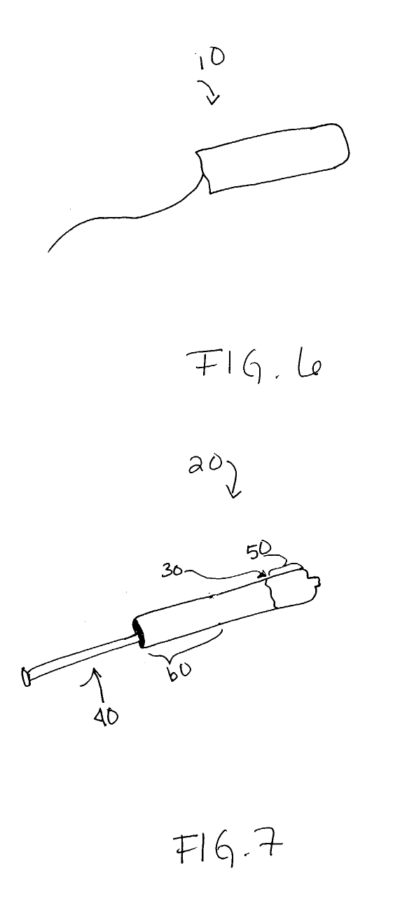

Another desirable embodiment provides for compositions of the invention in combination with a tampon (10; FIG. 6). For example, an enzyme, such as alcohol oxidase, is applied to tampon material as a solution and dried on the fibers. Alternatively, the enzyme may be dried first and then applied to the tampon. During use, the enzyme is rehydrated by the moisture in the vaginal tract, thus activating the enzyme. Alternatively, the tampon may be rehydrated with a solution prior to administration to the vagina. The enzyme may then act on the tampon material as a substrate, a separate substrate could be placed on the tampon, or the enzyme may use a substrate already present in the vagina for the production of peroxide in the vagina.

The enzymes and/or substrates useful in the present invention may be tethered or otherwise bound to an article or carrier/delivery vehicle. In one embodiment, the enzyme and/or substrate can be removed at will, ending the generation of peroxide (e.g., removal of a tampon that includes a substrate and/or an enzyme that produces a peroxide), and thus, ending treatment.

One embodiment of the invention provides for the use of a carrier that acts both to deliver the enzyme to the vaginal tract and as a substrate for those enzymes. In this embodiment of the invention, the substrate of the enzyme that catalyzes the production of peroxide is also the carrier that delivers the enzyme to the vaginal tract. For example, alcohol oxidase can be dried and placed on a poly (vinyl alcohol) support. This support is rehydrated prior to being placed in the vagina. The rehydrated enzyme oxidizes the poly (vinyl alcohol), in the presence of water and oxygen, to give hydrogen peroxide and oxidized poly (vinyl alcohol). Another example is an aqueous gel containing a mucoadhesive material, such as carboxymethylcellulose (optionally mixed with a thermogelling mucoadhesive agent), to be mixed with the enzyme just prior to use or as part of the dispensing process. The rehydrated enzyme then oxidizes the carrier, the carboxymethyl cellulose in the gel, and generates peroxide. An additional embodiment provides for the encapsulation of the enzyme in polymeric microparticles. Once in situ, the polymer, which contains alcohol moieties, is oxidized by the enzyme. In this case, release of the enzyme can be controlled by the microparticles to provide extended production of the desired product (e.g., sustained release). One example of a carrier or article to deliver the enzyme and act as a substrate is a tampon. Tampons contain cellulosic material or material, including, but not limited to, rayon or cotton. Cellulosic material contains alcohol groups that can be oxidized by oxidoreductase enzymes of the present invention, including alcohol oxidase, to yield peroxide. The combined enzyme delivery vehicle and substrate is not limited to peroxide generation in the vagina, but could also be applied to a wide variety of biomedical applications where delivery of an enzyme and the product of the enzyme are desired. Appropriate engineering of the substrates so they can be processed into delivery vehicles for the enzyme is required and would be recognized by those of skill in the art.

Additionally, separate from enzymatic activity, the delivery/substrate materials may yield degradation products that alter the vaginal environment in a beneficial manner. This class of materials may consist of backbone polymers that can be acted upon by an enzyme, onto which are grafted pendant chains of oligomers which can be hydrolytically degraded into an acid or acid producing species. The pendant chains may or may not serve as a substrate for the enzyme. For example, a poly (vinyl alcohol) backbone with pendant polycaprolactone chains, yielding poly [vinyl (polycaprolactate)] can be generated. The poly (vinyl alcohol) can serve as a substrate for alcohol oxidase to produce hydrogen peroxide. The polycaprolactone will hydrolytically degrade into caproic acid. This acid aids in lowering pH and controlling harmful bacterial growth, thus helping to restore balance to the vaginal system. In addition, this material is melt processable and can be formed into a system for controlled delivery of the enzyme. Additionally, a peroxide of Laureth-4 (e.g., a Laureth-4 terminal peroxide) would release laureth-4 and peroxide (e.g., hydrogen peroxide). Laureth-4 decreases TSS-1 production by S. aureus and the peroxide is available to suppress undesirable anaerobes and Gardneralla vaginalisis, thus reducing toxin production while reestablishing the vaginal flora.

B. Amount of Enzyme/Substrate

In order to be effective, the amount of enzyme and/or substrate in the compositions of the invention is an amount that is sufficient to restore or maintain a healthy homeostasis of vaginal bacterial flora or to inhibit the growth of microorganisms causing vaginal infection, such as bacterial vaginosis, while allowing beneficial microorganisms to prosper. Such an amount is determinable by a person of skill in the art.

For example, the compositions of the invention can be administered in unit dosage form, for example, containing 0.5 to 1000 International Units (IU), conveniently 2 to 500 IU, most conveniently, 10 to 100 IU of enzyme per unit dosage form. For example, a unit dosage form can contain about 1 unit, 2 units, 3 units or more units of enzyme per about 0.5 ml of a pharmaceutically acceptable carrier. The concentration of enzyme may be conveniently varied to accommodate varying volumes of mucus/fluid in the vaginal tract. Delivery can be easily achieved in a total volume of about 0.1 ml, 0.5 ml, 1 ml, 2 ml, 3 ml, 4 ml, 5 ml or more of a pharmaceutically acceptable carrier. The oxidizable substrate is generally present in the therapeutic or prophylactic composition in an amount from about 0.05% to about 99% by weight of the composition, such as an amount from about 0.2% to about 10% by weight of the composition. By varying the concentration of enzymes and substrates, the level of peroxide production can be controlled.

The exact regimen for administration of the compositions of the invention will necessarily be dependent upon the needs of the individual subject being treated and the type of treatment. For example, effective amounts of enzyme to treat a disturbance of the vaginal flora, including an infection, depends in part on whether the peroxide will be used therapeutically or prophylactically, the composition and severity of the disturbance or infection and/or the nature of the infection, the size and weight of the individual, etc. For example, during menstruation it may be desirable to deliver a composition that yields more hydrogen peroxide than during non-menstruating times. Determining an appropriate regimen is within the skill of the artisan.

The duration for use of the composition containing the enzyme also depends on whether the use is for prophylactic purposes, wherein the use may be, for example, daily or weekly, for a defined period of time, e.g., for a week, month or longer, or whether the use will be for therapeutic purposes, wherein a more intensive regimen of the use of the composition likely would be employed for a period of, for example, 2, 3, 4, 5 6, 7, 10, or 14 days or longer. Thus, therapeutic treatments could span several days or weeks, likely on a daily basis, and possibly at multiple intervals during the day. For example, the desired dose may conveniently be presented in a single dose or as divided doses administered at appropriate intervals, for example, as two, three, four or more sub-doses per day. The subdose itself may be further divided, e.g., into a number of discrete loosely spaced administrations.

In practice, the duration of exposure to active enzyme units will likely influence the desired concentration of active enzyme units employed in the dose. For example, carriers that are considered to provide prolonged release could provide a lower concentration of active enzyme units per dose, but over a longer period of time. Conversely, a shorter duration treatment, such as a douche, could provide a higher concentration of active units per dose. Any dosage form containing sufficient enzyme to provide effective concentrations of active enzyme at the site of the infection or to provide a sufficient prophylactic effect are well within the bounds of routine experimentation and therefore, well within the scope of the instant invention.

The compositions of the present invention may be administered to treat disturbances of the vaginal microflora or infection directly, after primary treatment, such as antibiotics or antifungals, or prophylactically, such as during menopause to give the lactobacilli “a boost.”

### IV. EXAMPLES

The following examples are meant to exemplify the present invention and should not be construed as limiting:

A. Peroxide Generation

Seven enzymes, including glucose oxidase, glycerol-3-phosphate oxidase, xanthine oxidase, cholesterol oxidase, galactose oxidase, alcohol oxidase and pyruvate oxidase, were tested for hydrogen peroxide generation on five substrates, including tampon material (e.g., cellulosic materials, including, but not limited to, cotton and rayon fibers), polydextran, carboxymethylcellulose, chitosan, and poly (vinyl alcohol).

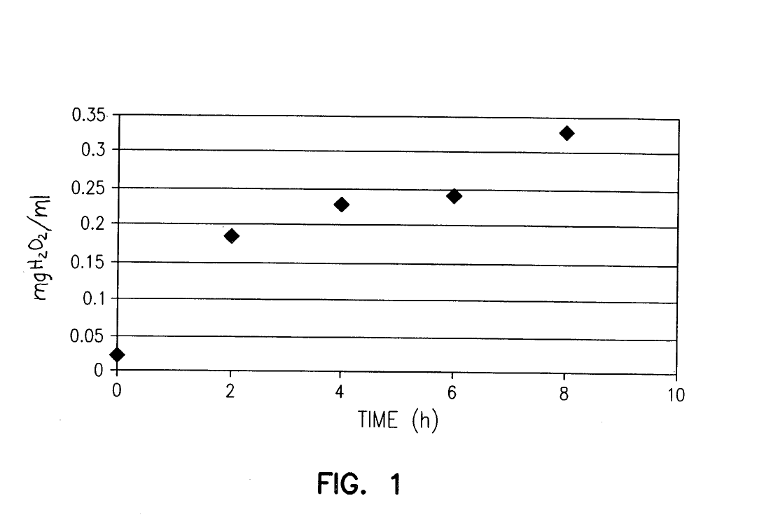

One unit of each enzyme was combined with 0.05 ml of a saturated solution (in distilled, deionized water) of each substrate in a test tube and diluted to 0.5 ml with buffer (0.5 M Tris buffer, pH adjusted to 6.3). In the case of insoluble substrates, a piece (approximately 0.25 g) of the substrate was placed in the test tube. The tubes were placed in an oscillating incubator at 37° C. After 12 h, the tubes were removed and the solution was assayed for H₂O₂ level by an established method (Graf, E. and Penniston, J. T., “Method for Determination of Hydrogen Peroxide, With Its Application Illustrated by Glucose Assay,” Clinical Chemistry, 26, 658-660 (1980)). The results are depicted in Table 2 in μg H₂O₂ per ml. (For production of hydrogen peroxide over time, see FIG. 1.)

TABLE 2
μg of H₂O₂
produced/ml

| | | | | | | | |
|---|---|---|---|---|---|---|---|
| Tampon | 4.43 | 4.84 | 10.46 | 4.92 | 6.45 | 97.84 | 6.66 |
| Material | | | | | | | |
| (cotton) | | | | | | | |
| Polydextran | 54.69 | 4.01 | 5.96 | 3.89 | 3.89 | 92.89 | 9.75 |
| CM Cellulose | 3.31 | 3.97 | 6.08 | 4.01 | 4.43 | 103.96 | 9.84 |
| Chitosan | 10.66 | 9.01 | 10.00 | 14.09 | 8.06 | 9.38 | 11.94 |
| Poly(vinyl | 4.47 | 3.85 | 6.00 | 5.13 | 4.34 | 53.20 | 3.85 |
| alcohol) | | | | | | | |
| | glucose | Glycerol-3- | xanthine | cholesterol | galactose | alcohol | pyruvate |
| | oxidase | phosphate | oxidase | oxidase | oxidase | oxidase | oxidase |
| | | oxidase | | | | | |

Alcohol oxidase had the highest level of H₂O₂ production on most of the substrates.

Modifications were performed on poly (vinyl alcohol) to improve the processability and increase the acid-delivering capabilities of poly (vinyl alcohol). These modifications included reacting poly (vinyl acetate) with poly (caprolactone) to yield a carbon backbone decorated with poly (caprolactone) chains. The method used to generate this material was as follows: to a solution of 4 parts poly (vinyl acetate) in toluene was added 1 part polycaprolactone and a catalytic amount of p-toluenesulfonic acid. The resulting solution was heated to reflux and the liberated acetic acid was removed via a Dean-Stark trap filled partially with a 10% solution of sodium bicarbonate. When the liberation of acetic acid ceased, the reaction was cooled and washed with water. The toluene was removed under vacuum to yield the modified pva/polycap polymer.

Enzyme action on the poly (caprolactone) chains could occur at the alcohol ends, and at the alcohols produced in the hydrolysis of the caprolactone ester bonds, which also yields an acid. Alcohol oxidase produced 22% more H₂O₂ on this material than on unmodified poly (vinyl alcohol) in a direct comparison (data not shown).

B. Effectiveness of H₂O₂

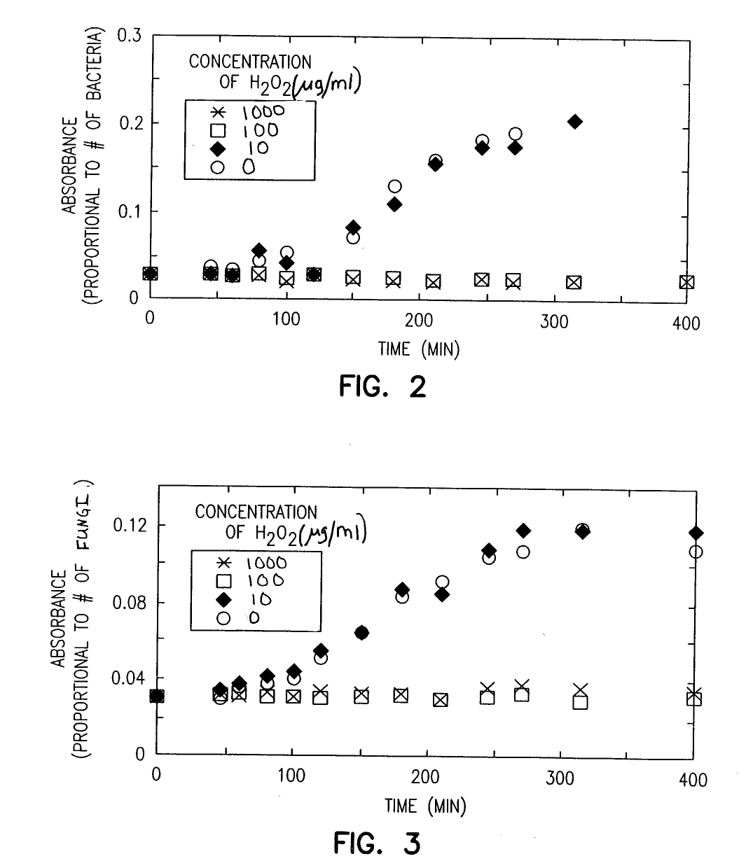

The effectiveness of H₂O₂ on suppressing the growth of pathogenic microbes, such as C. albicans and E. coli, was tested. Pathogenic microbes were subcultured in the presence of various concentrations of H₂O₂, and their growth was followed using optical density (absorbance) at 595 nm (FIGS. 2 and 3). The values between which growth inhibition takes place (over a 6 hour period) for both microbes was about 0.01 and about 0.001% H₂O₂, which corresponds to the range of about 10 to about 100 μg H₂O₂ per ml in Table 2 (FIGS. 2 and 3). These data indicate that the generation of H₂O₂ by enzymes can produce H₂O₂ levels that have a therapeutic benefit.

C. Self-Limiting features of Peroxide Generation

One of the enzyme-substrate combinations, alcohol oxidase-carboxymethylcellulose, was further studied. One unit of enzyme was combined with 0.05 ml of 2.5% carboxymethyl cellulose in a test tube and diluted to 0.5 ml with buffer of appropriate pH. The tubes were placed in an oscillating incubator at 37° C. After 8 hours, the tubes were removed and the solution was assayed for H₂O₂ level.

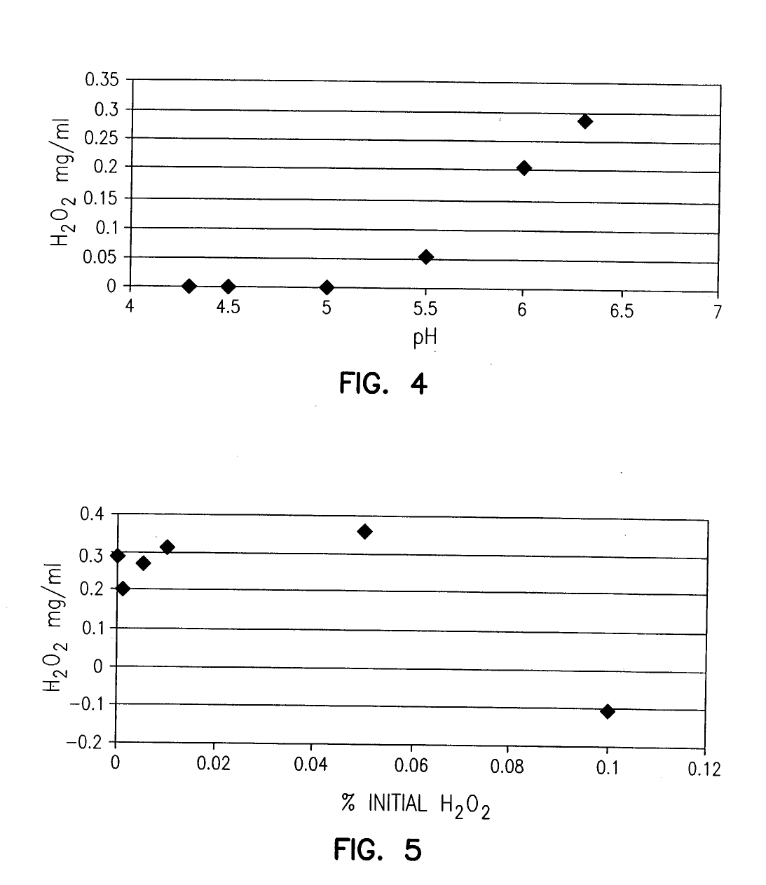

In the pathogenic (infected) state, the vaginal pH of 6 to 7 is higher than that of the healthy state (pH 4 to 4.5). Alcohol oxidase produced high levels of hydrogen peroxide at pH 6.3. However, the activity of alcohol oxidase decreased with decreasing pH (FIG. 4). Thus, if an enzyme and substrate are placed into an infected vaginal system with elevated pH, the enzyme will produce hydrogen peroxide. This hydrogen peroxide will inhibit pathogenic microorganisms and allow the beneficial microorganism(s) to proliferate. As a healthy vaginal homeostasis is reestablished, the pH will drop and the activity of the enzyme, e.g., alcohol oxidase, will fall. This corresponds with the reduction in need for hydrogen peroxide generation, and effectively ensures that hydrogen peroxide will not be generated when it is not needed.

Another advantage of the present system is that certain enzymes, such as alcohol oxidase, are deactivated in the presence of high concentrations of hydrogen peroxide (FIG. 5). This feature prevents the overproduction of hydrogen peroxide, and keeps levels below the concentration harmful to beneficial bacteria and vaginal tissues.

All cited publications, patents, and patent applications are incorporated by reference herein, as though individually incorporated by reference. The invention has been described with reference to various embodiments and techniques. However, it should be understood that many variations and modifications may be made while remaining within the spirit and scope of the invention.

What is claimed is:

1. A composition for use in the therapeutic or prophylactic treatment of a disturbance of vaginal bacterial flora in mammals comprising vaginal administration of an effective amount of a composition comprising an enzyme capable of producing peroxide upon administration of said composition to the vagina of said mammal.

2. The composition of claim 1, wherein the enzyme comprises an oxidoreductase, lipoxygenase, or prostaglandin synthase enzyme.

3. The composition of claim 2, wherein the oxidoreductase enzyme comprises an enzyme selected from the group consisting of malate oxidase, glucose oxidase, hexose oxidase, cholesterol oxidase, aryl-alcohol oxidase, 1-gulonolactone oxidase, galactose oxidase, pyranose oxidase, 1-sorbose oxidase, pyridoxine 4-oxidase, alcohol oxidase, catechol oxidase, (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, xanthine oxidase, thiamine oxidase, 1-galactonolactone oxidase, cellobiose oxidase, columbamine oxidase, hydroxyphytanate oxidase, nucleoside oxidase, N-acylhexosamine oxidase, polyvinyl-alcohol oxidase, methanol oxidase, D-arabinono-1,4-lactone oxidase, vanillyl-alcohol oxidase, D-mannitol oxidase, and mixtures thereof.

4. The composition of claim 2, wherein the oxidoreductase enzyme comprises glucose oxidase, glycerol-3-phosphate oxidase, xanthine oxidase, cholesterol oxidase, galactose oxidase, alcohol oxidase or pyruvate oxidase.

5. The composition of claim 2, wherein the oxidoreductase enzyme comprises alcohol oxidase.

6. The composition of claim 1, wherein the enzyme comprises a self-limiting enzyme.

7. The composition of claim 1, wherein the enzyme is deactivated in presence of high levels of peroxide or low pH.

8. The composition of claim 1 further comprising a substrate that said enzyme is capable of acting on to produce peroxide.

9. The composition of claim 8, wherein the substrate comprises an oxidizable substrate.

10. The composition of claim 9, wherein the oxidizable substrate comprises (S)-malate, β-D-glucose, D-galactose, D-mannose, maltose, lactose, cellobiose, cholesterol, aromatic primary alcohol, primary alcohol, L-gulono-1,4-lactone, D-galactose, D-xylose, L-sorbose, D-glucono-1,5-lactone, pyridoxine, catechol, (S)-2-hydroxy acid, ecdysone, choline, secondary alcohol, (S)-2-hydroxy-2-(4-hydroxyphenyl)acetate, long-chain alcohol, long-chain fatty alcohol, sn-glycerol 3-phosphate, xanthine, hypoxanthine, thiamine, L-galactono-1,4-lactone, L-galactonic-1,4-lactone, D-altronic-1,4-lactone, L-fuconic-1,4-lactone, D-arabinic-1,4-lactone, D-threonic acid-1,4-lactone, cellodextrin, cellulose, lactose, 4-β-D-glucosyl-D-mannose, columbamine, L-2-hydroxyphytanate, inosine, adenosine, nucleoside, 2′-deoxynucleosides, arabinosides, N-acetyl-D-glucosamine, N-glycolylglucosamine, N-acetylgalactosamine, N-acetylmannosamine, polyvinyl alcohol, methanol, aliphatic alcohol, D-arabinono-1,4-lactone, vanillyl alcohol, mannitol, D-arabinitol, D-glucitol, or mixtures thereof.

11. The composition of claim 9, wherein the oxidizable substrate comprises cellulose, polydextran, carboxymethylcellulose, chitosan, or poly(vinyl alcohol).

12. The composition of claim 9, wherein the oxidizable substrate comprises poly (vinyl alcohol), a poly (vinyl alcohol) derivative including poly (vinyl acetate) that has been reacted with poly (caprolactone), or a combination thereof.

13. The composition of claim 8, wherein the substrate comprises cellulose, polydextran, carboxymethylcellulose, chitosan, poly (vinyl alcohol), or a poly(vinyl alcohol) derivative including poly(vinyl acetate) that has been reacted with poly (caprolactone) and the enzyme comprises alcohol oxidase.

14. The composition of claim 8, wherein the substrate comprises poly (vinyl alcohol), or a poly (vinyl alcohol) derivative including poly (vinyl acetate) that has been reacted with poly (caprolactone) and the enzyme comprises alcohol oxidase.

15. The composition of claim 1 further comprising a carrier for delivering said composition to the vagina.

16. The composition of claim 15, wherein the carrier comprises a vaginal insert, tablet, suppository, pessary, powder, talc or other solid, solution, liquid, spray, aerosol, douche, ointment, tampon, syringe-like applicator, foam, cream, gel, bioadhesive gel, paste, microcapsule, vaginal sponge, vaginal ring, controlled sustained release compositions, or a combination thereof.

17. The composition of claim 15, wherein the enzyme is tethered to the carrier.

18. The composition of claim 17, wherein the carrier comprises a tampon.

19. The composition of claim 15, wherein the carrier is also a substrate that said enzyme is capable of acting on to produce peroxide.

20. The composition of claim 19, wherein the carrier and substrate comprise tampon material.

21. The composition of claim 19, wherein the carrier and substrate comprise poly(vinyl alcohol), a poly(vinyl alcohol) derivative including poly(vinyl acetate) that has been reacted with poly (caprolactone), a gel containing a mucoadhesive material, or a combination thereof.

22. The composition of claim 8 or 15, wherein the enzyme, substrate or carrier generates a beneficial agent other than peroxide.

23. The composition of claim 22, wherein the beneficial agent comprises an acid.

24. The composition of claim 1, further comprising an acid, antibiotic, anesthetic, defensin, or antifungal.

25. The composition of claim 1, wherein the mammal is a human.

26. The composition of claim 8 comprising about 0.5 to about 1000 units of enzyme and about 0.05% to about 99% by weight of substrate.

27. A method for treating or preventing a disturbance of vaginal bacterial flora in mammals comprising vaginal administration of an effective amount of a composition comprising an enzyme capable of producing peroxide upon administration of the composition to the vagina of said mammal.

28. The method of claim 27, wherein the enzyme is an oxidoreductase enzyme.

29. The method of claim 28, wherein the oxidoreductase enzyme comprises an enzyme selected from the group consisting of malate oxidase, glucose oxidase, hexose oxidase, cholesterol oxidase, aryl-alcohol oxidase, 1-gulonolactone oxidase, galactose oxidase, pyranose oxidase, 1-sorbose oxidase, pyridoxine 4-oxidase, alcohol oxidase, catechol oxidase, (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, xanthine oxidase, thiamine oxidase, 1-galactonolactone oxidase, cellobiose oxidase, columbamine oxidase, hydroxyphytanate oxidase, nucleoside oxidase, N-acylhexosamine oxidase, polyvinyl-alcohol oxidase, methanol oxidase, D-arabinono-1,4-lactone oxidase, vanillyl-alcohol oxidase, D-mannitol oxidase, and mixtures thereof.

30. The method of claim 28, wherein the oxidoreductase enzyme comprises glucose oxidase, glycerol-3-phosphate oxidase, xanthine oxidase, cholesterol oxidase, galactose oxidase, alcohol oxidase or pyruvate oxidase.

31. The method of claim 28, wherein the oxidoreductase enzyme comprises alcohol oxidase.

32. The method of claim 27, wherein the enzyme is a self-limiting enzyme.

33. The method of claim 27, wherein the enzyme is deactivated in presence of high levels of peroxide or low pH.

34. The method of claim 27 further comprising a substrate that said enzyme is capable of acting on to produce peroxide

35. The method of claim 34, wherein the substrate comprises an oxidizable substrate.

36. The method of claim 35, wherein the oxidizable substrate comprises (S)-malate, β-D-glucose, D-galactose, D-mannose, maltose, lactose, cellobiose, cholesterol, aromatic primary alcohol, primary alcohol, L-gulono-1,4-lactone, D-galactose, D-xylose, L-sorbose, D-glucono-1,5-lactone, pyridoxine, catechol, (S)-2-hydroxy acid, ecdysone, choline, secondary alcohol, (S)-2-hydroxy-2-(4-hydroxyphenyl)acetate, long-chain alcohol, long-chain fatty alcohol, sn-glycerol 3-phosphate, xanthine, hypoxanthine, thiamine, L-galactono-1,4-lactone, L-galactonic-1,4-lactone, D-altronic-1,4-lactone, L-fuconic-1,4-lactone, D-arabinic-1,4-lactone, D-threonic acid-1,4-lactone, cellodextrin, cellulose, lactose, 4-β-D-glucosyl-D-mannose, columbamine, L-2-hydroxyphytanate, inosine, adenosine, nucleoside, 2′-deoxynucleosides, arabinosides, N-acetyl-D-glucosamine, N-glycolylglucosamine, N-acetylgalactosamine, N-acetylmannosamine, polyvinyl alcohol, methanol, aliphatic alcohol, D-arabinono-1,4-lactone, vanillyl alcohol, mannitol, D-arabinitol or D-glucitol.

37. The method of claim 35, wherein the oxidizable substrate comprises cellulose, polydextran, carboxymethylcellulose, chitosan, poly(vinyl alcohol), a poly(vinyl alcohol) derivative including poly(vinyl acetate) that has been reacted with poly (caprolactone), or a combination thereof.

38. The method of claim 35, wherein the oxidizable substrate comprises poly(vinyl alcohol), a poly(vinyl alcohol) derivative including poly(vinyl acetate) that has been reacted with poly (caprolactone), or a combination thereof.

39. The method of claim 34, wherein the substrate comprises cellulose, polydextran, carboxymethylcellulose, chitosan, poly (vinyl alcohol), or a poly(vinyl alcohol) derivative including poly(vinyl acetate) that has been reacted with poly (caprolactone) and the enzyme comprises alcohol oxidase.

40. The method of claim 34, wherein the substrate comprises poly (vinyl alcohol), or a poly (vinyl alcohol) derivative including poly (vinyl acetate) that has been reacted with poly (caprolactone) and the enzyme comprises alcohol oxidase.

41. The method of claim 27 further comprising a carrier to deliver said composition to the vagina.

42. The method of claim 41, wherein the carrier comprises a vaginal insert, tablet, suppository, pessary, powder, talc or other solid, solution, liquid, spray, aerosol, douche, ointment, tampon, syringe-like applicator, foam, cream, gel, bioadhesive gel, paste, microcapsule, vaginal sponge, vaginal ring, controlled, sustained release compositions, or a combination thereof.

43. The method of claim 41, wherein the enzyme is tethered to the carrier.

44. The method of claim 43, wherein the carrier comprises a tampon.

45. The method of claim 44, wherein removal of the tampon from the vagina results in termination of treatment.

46. The method of claim 41, wherein the carrier is also a substrate that said enzyme is capable of acting on to produce peroxide.

47. The method of claim 46, wherein the carrier and substrate comprise tampon material.

48. The method of claim 46, wherein the carrier and substrate comprise poly (vinyl alcohol), poly (vinyl alcohol) derivative including poly (vinyl acetate) that has been reacted with poly (caprolactone), gel containing a mucoadhesive material, or a combination thereof.

49. The method of claim 34 or 41, wherein the enzyme, substrate or carrier generates a beneficial agent other than peroxide.

50. The method of claim 49, wherein the beneficial agent is an acid.

51. The composition of claim 27, further comprising an acid, antibiotic, anesthetic, or antifungal.

52. The method of claim 27, wherein the disturbance of vaginal bacterial flora is a reduction of the number of Gram-positive bacilli or a increase in the number of Gram-negative bacilli as compared to a healthy vaginal environment.

53. The method of claim 27, wherein the disturbance of vaginal bacterial flora is a vaginal infection.

54. The method of claim 53, wherein the vaginal infection is of bacterial or fungal origin.

55. The method of claim 53, wherein the vaginal infection is a result of increased concentrations of Gram-negative bacilli as compared to a healthy vagina.

56. The method of claim 53, wherein the vaginal infection is a result of decreased concentrations of Gram-positive bacilli as compared to a healthy vagina.

57. The method of claim 53, wherein vaginal infection is bacterial vaginosis.

58. The method of claim 27, wherein the administration of the composition promotes growth of Gram-positive bacilli.

59. The method of claim 27, wherein the administration of the composition inhibits Gram-negative bacilli.

60. The method of claim 34, wherein the composition comprises about 0.5 to about 1000 units of enzyme and about 0.05% to about 99% by weight of substrate.

61. The method of claim 27, wherein the mammal is a human.

62. An article for use in the therapeutic or prophylactic treatment of a disturbance of vaginal bacterial flora in mammals comprising an enzyme capable of producing peroxide, the article being adapted for use in the vagina.

63. The article of claim 62 comprising a vaginal insert, tablet, suppository, pessary, powder, talc or other solid, solution, liquid, spray, aerosol, douche, ointment, tampon, syringe-like applicator, foam, cream, gel, bioadhesive gel, paste, microcapsule, vaginal sponge, vaginal ring, controlled sustained release compositions, or a combination thereof.

64. The article of claim 62 comprising a tampon.

65. The article of claim 64, wherein the tampon comprises cellulosic material.

66. The article of claim 65, wherein the cellulosic material comprises rayon, cotton or a combination thereof.

67. The article of claim 62 comprising a syringe-like applicator.

68. The article of claim 62 further comprising a substrate that said enzyme is capable of acting on to produce peroxide.

69. The article of claim 68 comprising a syringe-like applicator.

70. The article of claim 69, wherein the substrate and enzyme are both present in the syringe-like applicator.

71. The article of claim 70, wherein the substrate and enzyme are separated by a barrier in the syringe-like applicator.
